(19) United States
(12) Patent Application Publication
(10) Pub. No.: US 2010/0076385 A1
(43) Pub. Date: Mar. 25, 2010

(54) DISPOSABLE PROTECTIVE FILM FOR BLOOD SAMPLING DEVICE

(75) Inventors: Tong-Yuh Huang, Hsinchu City (TW); Chin-Chia Lin, Taoyuan County (TW); Hui-Fang Wang, Hsinchu City (TW)

Correspondence Address: EGBERT LAW OFFICES, 412 MAIN STREET, 7TH FLOOR, HOUSTON, TX, 77002, US

(73) Assignee: SAND COUNTY BIOTECHNOLOGY, Inc., Hsin-Chu Industrial Park (TW)

(21) Appl. No.: 12/237,353

(22) Filed: Sep. 24, 2008

(51) Int. Cl. A61M 5/32

(52) U.S. Cl. 604/263

(57) ABSTRACT

The present invention provides a disposable protective film for a blood sampling device. The disposable protective film is assembled onto the pricker perforating end of the blood sampling device. The disposable protective film includes a film surface and a locating portion. The film surface is made of soft material that can be pierced by the pricker of the blood sampling device. The film surface is attached onto the end surface of the pricker perforating end of the blood sampling device. The locating portion, located laterally onto the film surface, is positioned onto the pricker perforating end of the blood sampling device. The disposable protective film provides a protective isolation for the blood sampling device and efficiently prevents adhesion of blood and dirt onto the blood sampling device.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates generally to an accessory for a blood sampling device, and more particularly to an innovative accessory with a disposable protective film that can be assembled onto the perforating end of the pricker of the blood sampling device.

2. Description of Related Art

Including Information Disclosed Under 37 CFR 1.97 and 37 CFR 1.98.

The blood sampling device of the present invention is generally a lancing device (referred to as a blood sampling needle). The blood sampling device is provided with a pricker, which can pierce through the hole at the head of blood sampling device and rebound quickly via a switch, so a needle-like wound may formed on the skin of the user for easy blood sampling.

Due to direct contact of the blood sampling device with human blood, contamination and health are of utmost concern. As the motion of the pricker involves the interior and exterior of the blood sampling device, the head of the blood sampling device must contact human skin. The blood from the wound is likely exposed to the head of the blood sampling device, or the pricker, leading to contamination of the blood sampling device. No matter if the blood sampling device is used by a single user or several users, serious safety hazards occur due to lack of sanitation, an unavoidable problem for a blood sampling device.

Thus, to overcome the aforementioned problems of the prior art, it would be an advancement in the art to provide an improved structure that can significantly improve efficacy.

Therefore, the inventor has provided the present invention of practicability after deliberate design and evaluation based on years of experience in the production, development and design of related products.

### BRIEF SUMMARY OF THE INVENTION

Based on the innovative structure of the disposable protective film of the present invention, the disposable protective film provides a protective isolation for the blood sampling device. The adhesion of blood and dirt onto the blood sampling device is efficiently prevented, thereby improving the sanitation and safety with better applicability.

Although the invention has been explained in relation to its preferred embodiment, it is to be understood that many other possible modifications and variations can be made without departing from the spirit and scope of the invention as hereinafter claimed.

### BRIEF DESCRIPTION OF THE SEVERAL VIEWS OF THE DRAWINGS

FIG. 1 depicts an exploded perspective view of the disposable protective film and blood sampling device of the present invention.

FIG. 2 depicts an assembled sectional view of the disposable protective film and blood sampling device of the present invention.

FIG. 3 depicts a partial sectional and schematic view of the disposable protective film of the present invention.

FIG. 4 depicts an isolated elevation view of the application of another structure of the disposable protective film of the present invention.

FIG. 5 depicts a partial sectional and schematic view of an application of another structure of the disposable protective film of the present invention.

FIG. 6 depicts a sectional view of an application view of the present invention, wherein the film surface is additionally provided with pricker punch hole.

FIG. 7 depicts a partial sectional and schematic view of an application of the present invention that the locating portion of the disposable protective film has an adhesive surface.

### DETAILED DESCRIPTION OF THE INVENTION

The features and the advantages of the present invention will be more readily understood upon a thoughtful deliberation of the following detailed description of a preferred embodiment of the present invention with reference to the accompanying drawings.

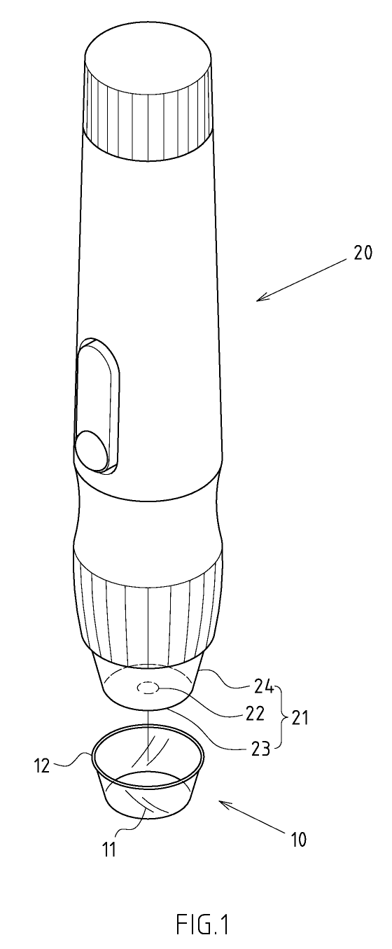

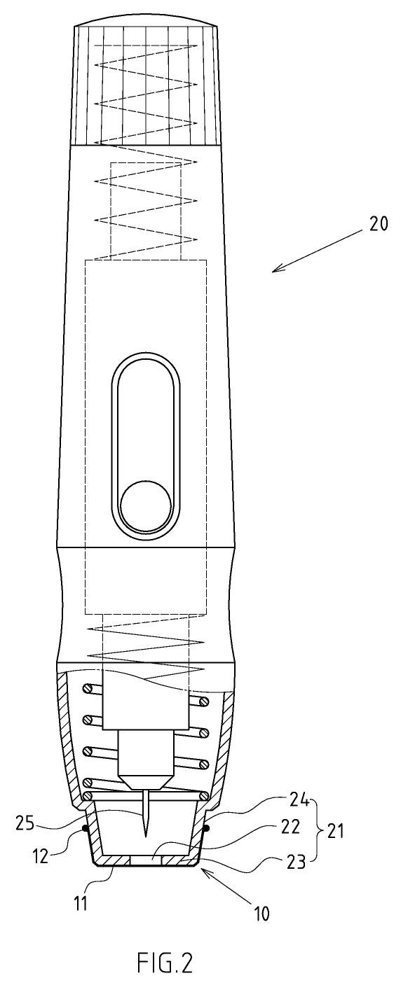

FIGS. 1-2 depict preferred embodiments of a disposable protective film for the blood sampling device of the present invention. The embodiments are only provided for explanatory purposes with respect to the patent claims.

The disposable protective film 10 is assembled onto the pricker perforating end 21 of existing blood sampling device 20. The pricker perforating end 21 comprises an end surface 23 with a puncture hole 22, as well as a circumferential portion 24. The puncture hole 22 is used for threading of the pricker 25 assembled into the blood sampling device 20.

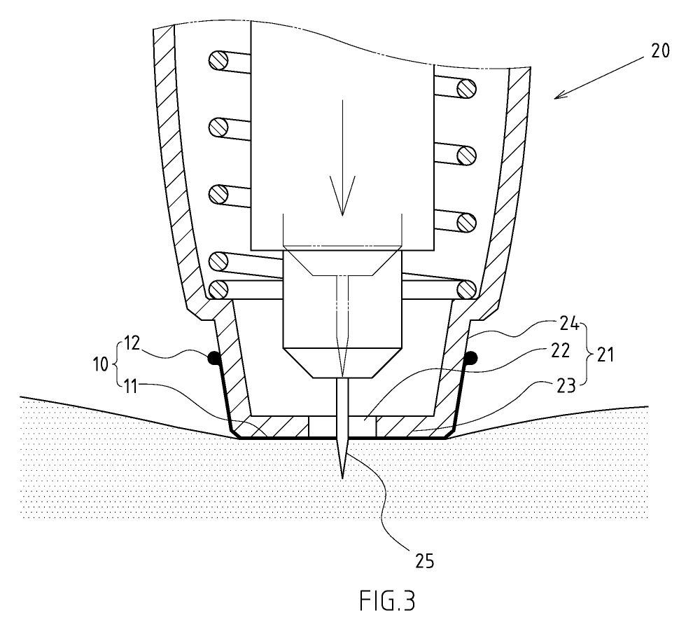

The disposable protective film 10 comprises a film surface 11, made of soft material that can be pierced by the pricker 25 of the blood sampling device 20 (as shown in FIG. 3). The film surface 11 is attached onto the end surface 23 of the pricker perforating end 21 of the blood sampling device 20.

The film 10 also includes a locating portion 12, located around the film surface 11. The locating portion 12 is sleeved onto the circumferential portion 24 of the pricker perforating end 21 of the blood sampling device 20, so as to locate firmly the disposable protective film 10.

Referring to FIGS. 1 and 2, the film surface 11 of the disposable protective film 10 can be a cup-shaped model in conformity with the pricker perforating end 21 of the blood sampling device 20. So, the film surface 11 can be attached onto the end surface 23 and circumferential portion 24 of the pricker perforating end 21 of the blood sampling device 20.

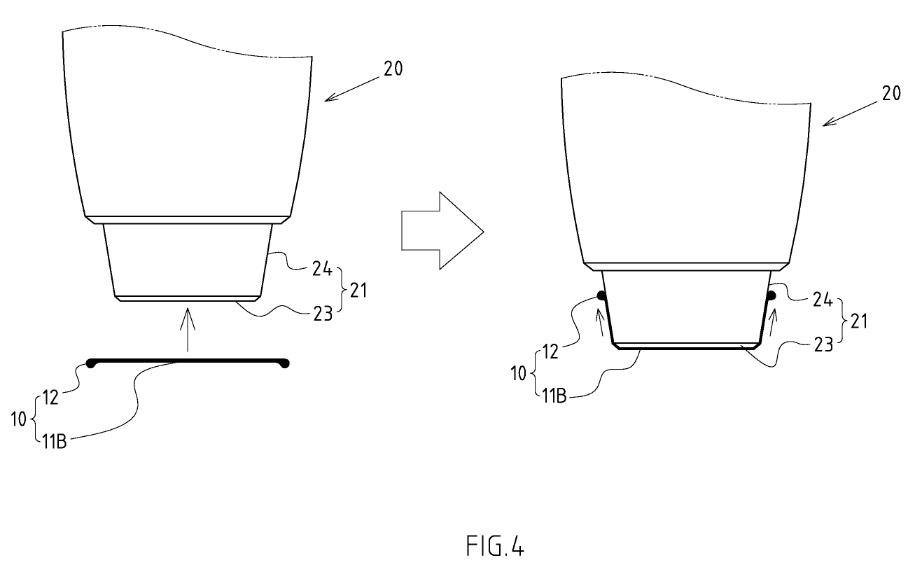

Referring to FIG. 4, the film surface 11B of the disposable protective film 10 can be flexibly retracted and designed into a flat model. After the film surface 11B is attached onto the end surface 23 of the pricker perforating end 21 of the blood sampling device 20, an upward pulling force is applied to extend and sleeve it onto the circumferential portion 24 of the pricker perforating end 21.

The locating portion 12 is a solid or a flexible ring body, so it can be bound flexibly in response to the dimensional change of the pricker perforating end 21 of the blood sampling device 20 (shown in FIG. 4).

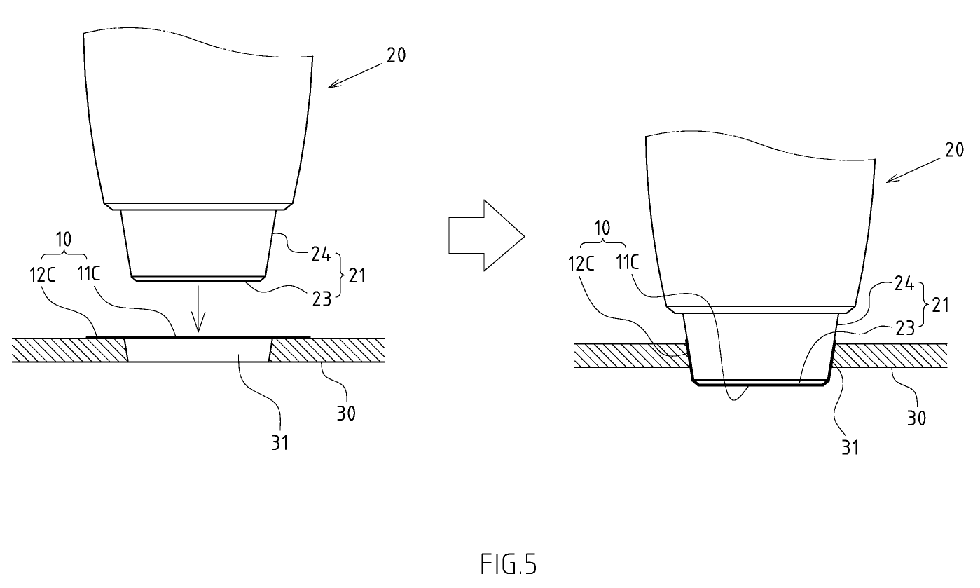

Referring to FIG. 5, the film surface 11C and locating portion 12C of the disposable protective film 10 are made of flexible film. When the film is assembled onto the pricker perforating end 21 of the blood sampling device 20, the disposable protective film 10 is flatly coupled with a jig 30 with interpolation hole 31. Then, the film surface 11C is aligned with the interpolation hole 31, and the pricker perforating end 21 of the blood sampling device 20 is inserted into the interpolation hole 31 of the jig 30. So, the film surface 11C is attached onto the end surface 23 of the pricker perforating end 21 of the blood sampling device 20, and the locating portion 12C is attached onto the circumferential portion 24 of the pricker perforating end 21.

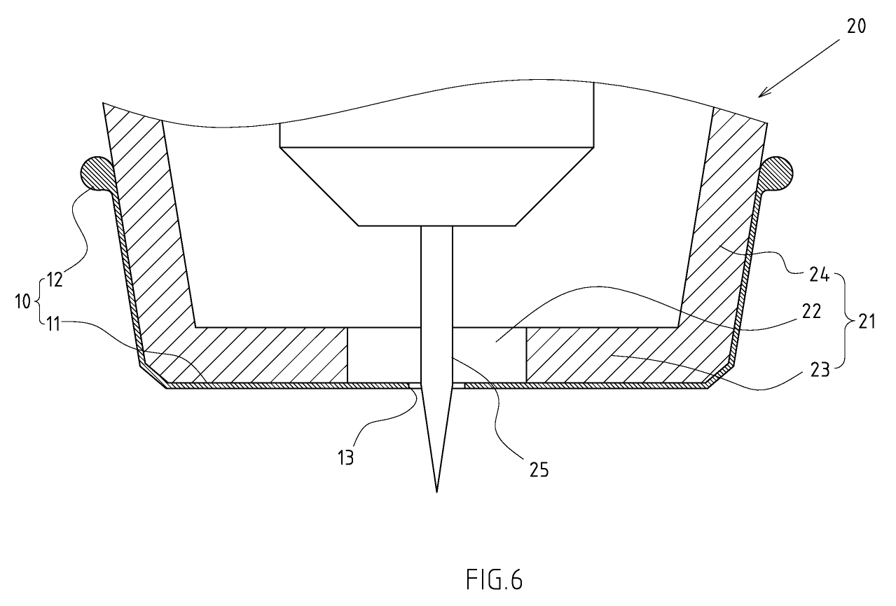

Referring also to FIG. 6, the film surface 11 of the disposable protective film 10 is provided with a pricker punch hole 13. The pricker punch hole 13 is located opposite to the pricker 25 of the blood sampling device 20, so that the pricker 25 could rightly penetrate through the pricker punch hole 13.

Based upon the above-specified structures, the disposable protective film 10 of the present invention is assembled onto the pricker perforating end 21 of existing blood sampling device 20 for protective isolation. Referring to FIG. 3, when the blood sampling device 20 is applied, the disposable protective film 10 is used for sleeving onto the end surface 23 and circumferential portion 24 of the pricker perforating end 21. When the pricker 25 of the blood sampling device 20 penetrates through the puncture hole 22 of the end surface 23 and pierces into the human skin, the bleeding blood and the dirt on the human skin are stuck onto the surface of the disposable protective film 10, rather than the blood sampling device 20. After completion of operation, the user is only required to peel off the disposable protective film 10 from the pricker perforating end 21 of the blood sampling device 20, thereby keeping blood sampling device 20 clean to maintain its operational safety.

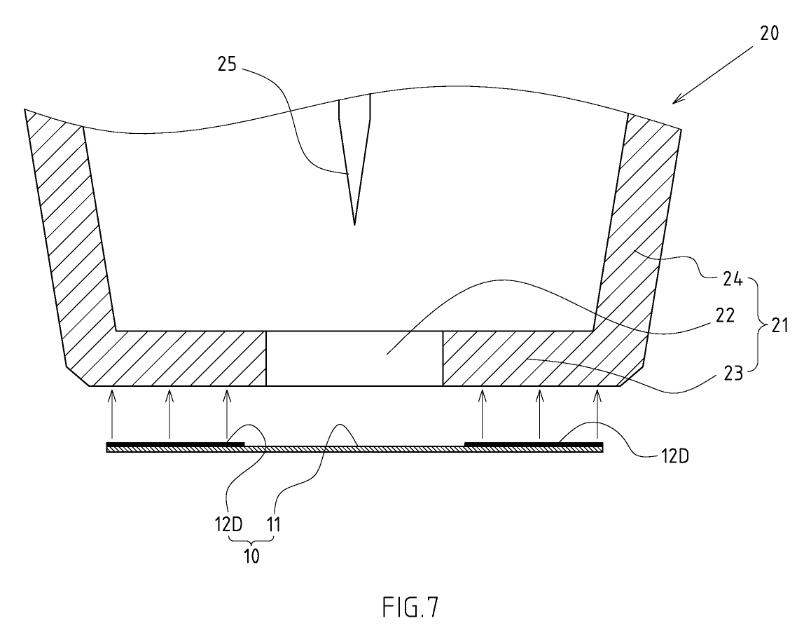

In addition, referring to FIG. 7, the locating portion 12D of the disposable protective film 10 has an adhesive surface that can be adhered partially or fully onto the surface of the disposable protective film 10, and located on the end surface 23 and/or circumferential portion 24 of the pricker perforating end 21.

1. A disposable protective film for a blood sampling device, said blood sampling device having a pricker, and a pricker perforating end with an end surface, a puncture hole, and a circumferential portion, said disposable protective film being assembled on said pricker perforating end, said disposable protective film comprising:
a film surface, being pierced by said pricker and being attached onto said end surface of said pricker perforating end of said blood sampling device; and
a locating portion, located laterally onto a preset position of said film surface said locating portion being positioned onto said pricker perforating end of the blood sampling device.

2. The film defined in claim 1, wherein said film surface is cup-shaped and attached onto said end surface and said circumferential portion of said pricker perforating end of said blood sampling device.

3. The film defined in claim 1, wherein said film surface is flexibly retracted.

4. The film defined in claim 1, wherein said locating portion is comprised of a solid ring body.

5. The film defined in claim 1, wherein said locating portion is comprised of a flexible ring body.

6. The film defined in claim 1, wherein the film surface and locating portion are comprised of flexible film, said film surface being flatly coupled with a jig with an interpolation hole, said film surface being aligned with said interpolation hole, said interpolation hole receiving said pricker perforating end of said blood sampling device, said film surface being attached onto said end surface of said pricker perforating end of the blood sampling device, said locating portion being attached onto said circumferential portion of said pricker perforating end.

7. The film defined in claim 1, wherein said film surface is provided with a pricker punch hole.

8. The film defined in claim 1, wherein said locating portion is arranged around said film surface, and sleeved onto said circumferential portion of said pricker perforating end of said blood sampling device.

9. The film defined in claim 1, wherein said locating portion is arranged laterally onto said film surface and adhered securely onto said end surface or said circumferential portion or both.
